(19) United States
(12) Patent Application Publication
(10) Pub. No.: US 2023/0398255 A1
(43) Pub. Date: Dec. 14, 2023

(54) CEILING STRUCTURE AND FACILITY COMPRISING SUCH A CEILING STRUCTURE COMPRISING AIR STERILISATION MEANS

(71) Applicants: Jean-Marc Scherrer, Riedisheim (FR); Damien Lang, Strasbourg (FR)

(72) Inventors: Jean-Marc Scherrer, Riedisheim (FR); Damien Lang, Strasbourg (FR)

(21) Appl. No.: 18/248,830

(22) PCT Filed: Oct. 13, 2021
(86) PCT No.: PCT/FR2021/051777

§ 371 Date: Apr. 12, 2023

(30) Foreign Application Priority Data
Oct. 13, 2020 (FR) FR2010441

(51) Int. Cl. A61L 9/20; E04B 9/02; E04B 9/04; E04B 9/30

(52) U.S. Cl. CPC A61L 9/20; E04B 9/02; E04B 9/04; E04B 9/303; A61L 2209/12; A61L 2209/15; A61L 2209/16

(57) ABSTRACT

The present disclosure relates to a false ceiling and an installation comprising such a structure for feeding treated air into a room, comprising a ceiling wall, a false wall positioned on the underside of, and at a distance from, all or a portion of the ceiling wall and defining, with the ceiling wall or a portion of the ceiling wall, an airflow corridor that is referred to as a plenum and comprises an air outlet opening inside the room provided in the false wall, characterized in that the false ceiling comprises lighting means comprising a plurality of light sources that are capable of emitting ultraviolet light and arranged in the airflow corridor so as to sterilize the air flowing in the corridor before it passes through the air outlet slot.

### CROSS-REFERENCE TO RELATED APPLICATIONS

This application is a national phase entry under 35 U.S.C. § 371 of International Patent Application PCT/FR2021/051777, filed Oct. 13, 2021, designating the United States of America and published as International Patent Publication WO 2022/079388 A1 on Apr. 21, 2022, which claims the benefit under Article 8 of the Patent Cooperation Treaty to French Patent Application Serial No. FR2010441, filed Oct. 13, 2020.

### TECHNICAL FIELD

The present disclosure relates to the field of ceilings bringing air, treated or not, into the associated premises. Treated air means heated, cooled, ventilated, dehumidified or humidified air.

The present disclosure relates more particularly to a ceiling structure and an installation comprising such a ceiling structure and making it possible to supply air treated in a room, comprising a ceiling wall, and a false wall positioned on the underside and at a distance from all or part of the ceiling wall and delimiting, with the ceiling wall or a portion of the ceiling wall, an air circulation corridor comprising an air outlet opening inside the room arranged in the false wall.

The ceiling structure and the associated installation according to the present disclosure are intended, in particular, but not exclusively for the sterilization of the air delivered in a room.

### BACKGROUND

Examples of embodiments of ceiling structures that make it possible to deliver treated air, and, in particular, ultraviolet-sterilized air, inside the associated room are known.

One can cite the application WO 2020/050864 A1 (SYNERGY MED GLOBAL DESIGN SOLUTIONS LLC A DELAWARE LLC [US]), which also discloses a ceiling installation in an operating room and intended to reduce the risk of patient infections (HAI), comprising structures connected to the ceiling, which make it possible to supply air curtains with laminar flow inside the room, to the interior of the room. In this installation, the air is sterilized in the technical ventilation part before it is injected directly inside the room. Access to the means involved in the sterilization of air, integrated with the means providing ventilation, is made difficult, or even delicate. Furthermore, the quality of the air sterilization depends on the exposure time of the latter within the ventilation unit. To increase the exposure time, it is necessary to reduce the aeraulic flow rate of the plant or to have very large air passage sections so as to reduce its speed. Effective air sterilization is therefore particularly difficult and expensive.

The patent application GB2127954 (WEISS TECH UMWELT KLIMA) may also be cited, which discloses an air supply ceiling for an operating room. The air supply ceiling sucks the supply air via an opening formed in the side wall of the room, located above a suspended ceiling of the section. The central section of the air supply ceiling comprises an air duct whose inlet opens inside the room and is supplied with air by a fan. The air inlet is provided with a sterilizing filter, through which the air is blown.

The air supply ceiling of the application GB2127954 has the disadvantage of ensuring only limited air sterilization due to treatment only when the filter passes through it, at the inlet of the air duct. Air sterilization is only ensured over a fraction of time corresponding to the time for which the filter passes through the air. Also, in this ceiling, the quality of the air sterilization depends on the flow rate of the air passing through the filter.

This air supply ceiling also has the disadvantage of imposing the location of the treatment system (air duct and filter) to that of the openings made in the false ceiling.

Furthermore, given the positioning of the processing system on the false ceiling as well as the space requirement thereof, the described installation can be extended to ceiling structures implementing tensioned fabrics or systems covering the ceiling surface.

The document FR 3 085 696 A1 (EGIS BATIMENTS [FR]; NORMALU [FR]) discloses a radiating technical ceiling allowing heating, air conditioning and ventilation of a room, characterized in that it comprises at least one tensioned fabric that allows the light and/or thermal radiation to pass completely or partially, the fabric (3) extending between walls and being arranged in order to define, with an upper slab, a space, and one or more radiating elements, formed of, for example, metal panels, and arranged to allow the circulation of a heat transfer fluid/coolant, the radiating elements being arranged in the space formed between the tensioned fabric (3) and the upper slab. The technical ceiling, according to its implementation, may be a false ceiling or an autonomous island.

The document KR 200 362 941 Y1 discloses an elevator lighting device, where the wind coming from the ventilation hole (20) passes through the ventilation guidance mechanism (30) and is blown into the elevator; this lighting device (40) comprises an ultraviolet sterilization lamp.

The document KR 2019 0061183 A (SILVER ELEVATOR KOREA CO LTD [KR]) describes an elevator involving an air sterilizer using ultraviolet (UV) rays and plasma. More specifically, a sterilizing elevator for hospital is proposed in order to prevent secondary infections in hospitals, one of the main sources of viruses and bacteria. To overcome this pollution, a clean elevator system is provided in order to manage the quality of the air thanks to efficient space sterilization. High-power UV technology is applied. Furthermore, thanks to fundamental prevention of infectious bacteria, the social impacts and economic losses may be prevented in advance by avoiding the propagation of a secondary infection in the hospital.

Document CN 105 756 256 B (ZHEJIANG FASHION BUILDING MAT CO LTD) describes an integrated ceiling with a disinfectant function. The integrated ceiling comprises a housing, wherein a lighting mechanism, a venting mechanism, a heating mechanism, a disinfection mechanism, and a controller are disposed in the housing; the lighting mechanism comprises a plurality of lamp tubes arranged in parallel, a transparent shade is disposed on the bottom surface of the housing and the plurality of lamp tubes is located on the interior side of the transparent map; the disinfection mechanism comprises a plurality of ultraviolet lamp tubes arranged in parallel, flaps are arranged on the bottom surface of the housing, are connected to a drive motor and comprise a plurality of blades arranged in a spliced manner, the surfaces of the blades are mirror surfaces, a plurality of through holes are formed uniformly in each blade, and the plurality of ultraviolet lamp tubes are located on the interior sides of the shutters; the ventilation mechanism comprises an air blow channel, and the plurality of ultraviolet lamp tubes are located in the air blowing channel, the external end opening of the air outlet tube forms an exhaust window, the plurality of ultraviolet lamp tubes are all located in the exhaust window, and an air blowing is arranged in the air blowing channel.

Document US 2019/292315 A1 (NIEMIEC DARRIN [US]; CARLSON WILLIAM [US]) describes a combined axial fan and a LED lighting system configured to adapt to the imprint of a standard ceiling slab. Described embodiments further comprise ceiling tiles with an integrated fan and/or LED illumination. The disclosed systems may include a housing container and an axial fan. The fan has a fan cavity comprising an air bypass mechanism for directing air from the fan cavity to the lighting and fan components. The present disclosure comprises an air circulation surface for directing existing air into the fan cavity along a LED light. In addition, the described embodiments comprise one or more UV light sources that irradiate contaminants when air flows through the ceiling slab. The system for drawing air from a room to be treated is done vertically at the fan; deflectors will orient the sucked-in air to be sterilized in a multidirectional manner toward side chambers or housings where a UV ray reflection system is arranged over the entire surface of the housing in order to improve the action of ultraviolet lamps.

The present disclosure aims to remedy these problems by proposing a ceiling structure making it possible to bring sterilized air, the sterilization quality of which is guaranteed, into a premises and improved relative to that proposed by the ceiling structures of the state of the art.

The purpose of the ceiling structure is also to ensure a controlled and regular sterilization of the air, the efficiency of which is much less related to the flow rate of the air to be treated.

The purpose of the ceiling structure is also to be implemented with a false ceiling, advantageously with a tensioned fabric, without harming the aesthetics of the latter.

### BRIEF SUMMARY

For this purpose, and according to a first aspect, the present disclosure proposes a ceiling structure making it possible to bring the treated air into a room, comprising a ceiling wall, a false wall positioned under and at a distance from all or part of the ceiling wall and delimiting, with the ceiling wall or a portion of the ceiling wall, an air circulation corridor comprising an air outlet opening inside the room arranged in the false wall. The ceiling structure comprises light means that comprise a plurality of light sources capable of emitting ultraviolet light arranged in the air circulation corridor so as to sterilize the air flowing in the corridor before it passes through the air outlet opening.

The present disclosure proposes to provide a false ceiling making it possible to supply treated or untreated air in a room to be sterilized, comprising:
at least one protective tensioned fabric, blocking UVC, with the protective tensioned fabric positioned on the underside and at a distance from the ceiling of the room to be sterilized, being fixed to the side walls of the room and arranged with an upper slab of the ceiling to delimit a so-called plenum space,
at least one means for attaching at least one protective tensioned fabric to the side walls of the room by means of at least one peripheral attachment profile comprising an air outlet slot opening onto at least one side wall of the room and allowing the passage of the air circulating between the plenum and the room to be sterilized,
at least one ventilation and/or air-conditioning means connected to the plenum via at least one air inlet ventilation duct (9) in the plenum, the blown air forming a horizontal unidirectional air circulation corridor over the entire length of the plenum,
an air return box able to be connected to ventilation and/or air-conditioning means, as well as air suction openings located opposite the air outlet slot and allowing air to pass from inside the room to the air return box, and where the false ceiling comprises a plurality of UVC light sources arranged in the plenum over the entire length of the horizontal unidirectional air circulation chute so as to sterilize the air flowing in the air circulation corridor before it passes through the air outlet slot and where the distance between the plurality of UVC light sources and the ceiling on the one hand and the distance between the plurality of UVC light sources and the at least one tensioned protective fabric on the other hand is at most 25 cm.

In particular, the present disclosure proposes to provide a false ceiling making it possible to supply or not treated air in a room to be sterilized, comprising:
at least one protective tensioned fabric, blocking UVC, with the protective tensioned fabric positioned on the underside and at a distance from the ceiling of the room to be sterilized, being fixed to the side walls of the room and arranged with an upper slab of the ceiling to delimit a so-called plenum space,
at least one tensioned finishing fabric, the tensioned fabric, visible from the room, being fixed to the side walls of the room,
at least one means for attaching at least one protective tensioned fabric and at least one tensioned finishing fabric to the side walls of the room by means of at least one peripheral attachment profile comprising an air outlet slot opening onto at least one side wall of the room and allowing the passage of the air circulating between the plenum and the room to be sterilized,
at least one ventilation and/or air-conditioning means connected to the plenum via at least one air inlet ventilation duct in the plenum, the blown air forming a horizontal unidirectional air circulation corridor over the entire length of the plenum,
an air return box able to be connected to ventilation and/or air-conditioning means, as well as air suction openings located opposite the air outlet slot and allowing air to pass from inside the room to the air return box, and where the false ceiling comprises a plurality of UVC light sources arranged in the plenum over the entire length of the horizontal unidirectional air circulation chute so as to sterilize the air flowing in the air circulation corridor before it passes through the air outlet slot and where the distance between the plurality of UVC light sources and the ceiling on the one hand and the distance between the plurality of UVC light sources and the at least one tensioned protective fabric on the other hand is at most 25 cm.

Advantageously, the plurality of light sources emits UVC.

Advantageously, the light means comprise a plurality of light sources distributed over all or part of the wall or the wall portion of the ceiling.

Advantageously, the plurality of light sources are arranged at mid-height of the plenum.

Advantageously, the false wall is a false tensioned fabric wall. In this case, and advantageously, the ceiling structure comprises means blocking the ultraviolet light emitted by the plurality of light sources so as to form a screen for protecting the tensioned fabric of the false wall. According to a preferred configuration, the ultraviolet light blocking means comprise an additional tensioned fabric arranged between the tensioned fabric of the false wall and the ceiling wall.

Advantageously, the ceiling structure further comprises an air return box able to be connected or connected fluidly to ventilation and/or air conditioning means, as well as air suction openings allowing air to pass from inside the room into the enclosure. Thus, in addition to the blowing of air sterilized inside the room, the ceiling structure ensures the return of air blown into ventilation and/or air-conditioning means, to which the air return box will be connected.

The present disclosure also relates to an installation allowing air treated in a room to be sterilized, comprising a false ceiling according to the present disclosure as described above, an air inlet in the plenum, the air outlet slot of the false wall being arranged at the opposite end of the air inlet, and
ventilation and/or air conditioning means connected to the air inlet in order to form a horizontal unidirectional air circulation corridor of the plenum.

According to an advantageous embodiment, the ventilation and/or air-conditioning means are also fluidly connected to the air return chamber.

### BRIEF DESCRIPTION OF THE DRAWINGS

Other features and advantages of the present disclosure will emerge from the following detailed description of the present disclosure with reference to the appended figures, and in which:

FIG. 1 represents a cross section of a false ceiling structure allowing the circulation of ambient air processed within a premises according to an exemplary embodiment of the present disclosure for which the light sources are installed against the ceiling of the part.

FIG. 2 represents a cross section of a false ceiling structure allowing the circulation of ambient air processed within a premises according to another embodiment of the present disclosure for which the light sources are installed at mid-height of the plenum.

FIG. 3 represents a top view, namely from the ceiling of a false ceiling structure allowing the circulation of an ambient air processed within a premises according to another embodiment of the present disclosure for which the light sources are installed in continuous or discontinuous lines.

FIG. 4 represents a top view, namely from the ceiling of a false ceiling structure allowing the circulation of ambient air processed within a premises according to another embodiment of the present disclosure for which the light sources are installed in discontinuous lines and where two lines of discontinuous light lamps are adjoining the arrangement of their UVC light sources is arranged in staggered rows.

FIG. 5 represents a horizontal section resulting from the result of the numerical simulations.

FIG. 6 represents the impact of the height of the plenum (4) on the disinfection quality.

FIG. 7 represents the degree of disinfection as percentage of the interior air of the room or local to be treated as a function of the operating time of the light sources.

### DETAILED DESCRIPTION

Although methods and materials similar or equivalent to those described herein may be used in practice, suitable methods and materials are described below. All publications, patent applications, patents and other references mentioned herein are incorporated by reference in their entirety. In addition, the materials, methods and embodiments described are merely illustrative and are not intended to be limiting.

In the event of conflict, the present description, including definitions, will prevail.

Unless otherwise defined, all technical and scientific terms used in the present document have the same meaning as generally understood by the person skilled in the art to whom the subject belongs. As used herein, the following definitions are provided to facilitate understanding of the present disclosure.

The term “comprise or comprises” is generally used in the sense of including, that is to say, to allow the presence of one or more characteristics or components.

As used in the description and claims, the singular forms “a” and “the” include plural references, unless otherwise indicated.

The term “treated air” means cooled or reheated air (so as to ensure the cooling or heating requirements of a room) and provided with an external new air supply so as to ensure the ventilation of hygienic air of the room.

As a general rule, “cold air” is understood to mean a temperature ranging up to 20° C. and “hot air” any temperature above 20° C.

The present disclosure proposes to provide a false ceiling (1) making it possible to supply or not treated air in a room to be sterilized, comprising:
at least one protective tensioned fabric (8), blocking UVC, the protective tensioned fabric (8) positioned on the underside and at a distance from the ceiling (3) of the room to be sterilized, being fixed to the side walls of the room and arranged with an upper slab of the ceiling (3) in order to delimit a so-called plenum (4) space,
at least one means for attaching the at least one protective tensioned fabric (8) to the side walls of the room by means of at least one peripheral attachment profile (12) comprising an air outlet slot (5) opening onto at least one side wall of the room and allowing the passage of the air circulating between the plenum (4) and the room to be sterilized,
at least one ventilation and/or air-conditioning means (10) connected to the plenum (4) via at least one air inlet air venting duct (9) in the plenum (4), the blown air forming a horizontal unidirectional air circulation corridor over the entire length of the plenum (4),
an air return box (15) able to be connected to ventilation and/or air conditioning means (10), as well as air suction openings (14) located opposite the air outlet slot (5) and allowing air to pass from inside the room to the air return box (15),
and where the false ceiling (1) includes a plurality of UVC light sources (6) arranged in the plenum (4) over the entire length of the horizontal unidirectional air circulation chute so as to sterilize the air flowing in the air circulation corridor before it passes through the air outlet slot (5) and where the distance between the plurality of UVC light sources (6) and the ceiling (3) on the one hand and the distance between the plurality of UVC light sources (6) and the at least one tensioned protective fabric (8) on the other hand is at most 25 cm.

Preferably, the false ceiling (1) according to the present disclosure further comprises at least one tensioned finishing fabric (2), the tensioned finishing fabric (2), visible from the room, being fixed to the side walls of the room by means of at least one peripheral attachment profile (12) comprising an air outlet slot (5) opening onto at least one side wall of the room and allowing the passage of the air circulating between the plenum (4) and the room to be sterilized.

Thus, according to a preferred embodiment, the present disclosure proposes to provide a false ceiling (1) making it possible to supply or not treated air in a room to be sterilized, comprising:
at least one protective tensioned fabric (8), blocking UVC, the protective tensioned fabric (8) positioned on the underside and at a distance from the ceiling (3) of the room to be sterilized, being fixed to the side walls of the room and arranged with an upper slab of the ceiling (3) in order to delimit a so-called plenum (4) space,
at least one tensioned finishing fabric (2), the tensioned fabric (2), visible from the room, being fixed to the side walls of the room,
at least one means for attaching the at least one protective tensioned fabric (8) and the at least one tensioned finishing fabric (2) to the side walls of the room by means of at least one peripheral attachment profile (12) comprising an air outlet slot (5) opening onto at least one side wall of the room and allowing the passage of the air circulating between the plenum (4) and the room to be sterilized,
at least one ventilation and/or air-conditioning means (10) connected to the plenum (4) via at least one air inlet air venting duct (9) in the plenum (4), the blown air forming a horizontal unidirectional air circulation corridor over the entire length of the plenum (4),
an air return box (15) able to be connected to ventilation and/or air conditioning means (10), as well as air suction openings (14) located opposite the air outlet slot (5) and allowing air to pass from inside the room to the air return box (15),
and where the false ceiling (1) includes a plurality of UVC light sources (6) arranged in the plenum (4) over the entire length of the horizontal unidirectional air circulation chute so as to sterilize the air flowing in the air circulation corridor before it passes through the air outlet slot (5) and where the distance between the plurality of UVC light sources (6) and the ceiling (3) on the one hand and the distance between the plurality of UVC light sources (6) and the at least one tensioned protective fabric (8) on the other hand is at most 25 cm.

Preferably, the height of the plenum (4) is at most 50 cm.

According to a particular embodiment, the height of the plenum (4) is at least 10 cm.

Advantageously, the plurality of UVC light sources (6) is arranged at mid-height of the plenum (4).

According to an embodiment, the plurality of UVC light sources (6) is arranged in the form of lines of light lamps (13) that are spaced apart over the entire length of the plenum (4).

According to another embodiment of the present disclosure, the lines of light lamps (13) are continuous or discontinuous lines (see FIG. 3).

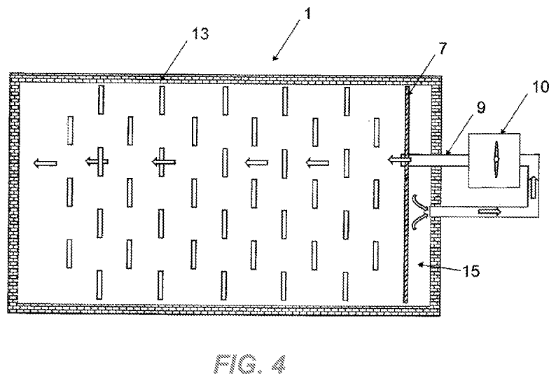

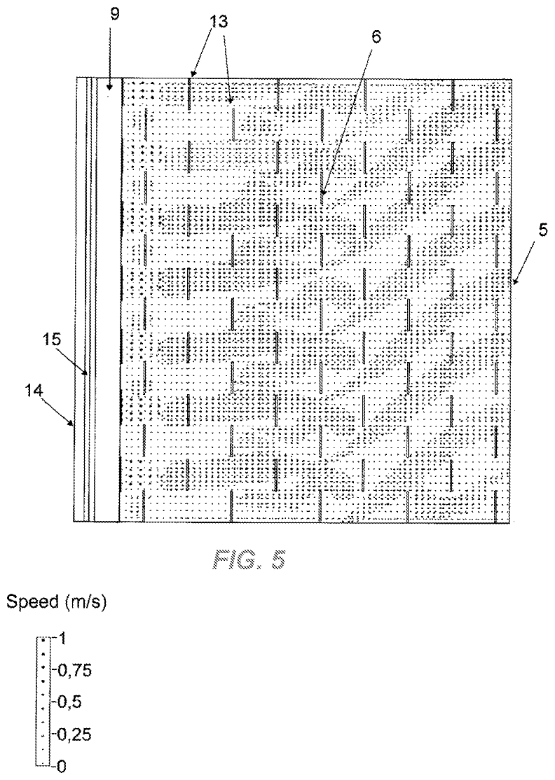

Preferably, in the case where two discontinuous lines of light lamps (13) are adjoining the arrangement of their UVC light sources (6) is arranged in staggered rows (see FIG. 4). The term “staggered” is understood to mean that the UVC light sources (6) of two lines of neighboring light lamps (13) alternate in two rows.

Advantageously, the space between two lines of light lamps (13) is between 25 cm and 200 cm.

Preferably, the space between two lines of light lamps (13) is between 50 cm and 100 cm.

Surprisingly, it has been demonstrated that the air of the room to be sterilized has a disinfection coefficient of 99.9% of the viruses and bacteria when the ventilation and/or air conditioning means (10) is activated for at most 30 minutes and this independently of the number of occupants in the room to be sterilized.

Furthermore, it has also been demonstrated that the air of the room to be sterilized has a disinfection coefficient of 99.9% of the viruses and bacteria when the ventilation and/or air conditioning means (10) is activated for at least 8 minutes and this independently of the number of occupants in the room to be sterilized.

Preferably, the walls of the plenum (4) comprise means for reflecting ultraviolet light emitted by the plurality of UVC light sources (6).

According to a preferred embodiment of the present disclosure, the face of the protective tensioned fabric (8) facing the plenum (4) has a reflection coefficient for UVC greater than or equal to 80%.

Advantageously, the protective tensioned fabric (8) and the finishing fabric (2) have a thickness of less than 5 mm.

According to another embodiment, the suction channel (15) is formed by a separation partition (7) integral with the peripheral attachment profile (12).

The present disclosure also proposes to provide an installation allowing air treated in a room to be sterilized, comprising:
a false ceiling (1) according to the present disclosure,
an air inlet (9) in the plenum (4), the air outlet slot (5) of the false wall being arranged at the opposite end of the air inlet (9), and
ventilation and/or air conditioning means (10) connected to the air inlet (9) in order to form a horizontal unidirectional air circulation corridor (4).

Advantageously, the ventilation and/or air conditioning means (10) are connected to the air return box (15).

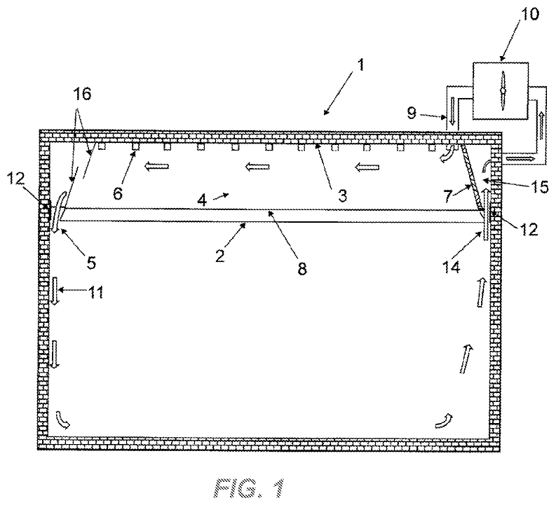

In relation to FIG. 1, a ceiling or false ceiling structure (1) is described that makes it possible to supply treated air (11) within a room. In the present disclosure, the treatment relates to the sterilization of air.

The false ceiling (1) comprises, in the shown example, a protective tensioned fabric (8), positioned on the underside of the ceiling wall (3). The protective tensioned fabric (8) extends between the walls of the room to be sterilized and is fixed to these walls by means of attachment profiles (12) (also called smooth). In the following, the false sheet wall is designated by first protective fabric (8) in order to simplify the reading of the present description.

The first tensioned fabric (8) forms a fabric that can be visible from inside the room. It is arranged so as to delimit, with the ceiling wall (3), an air circulation corridor (4) (also called plenum).

The false ceiling (1) comprises an air inlet or aeraulic ventilation (9) in the air circulation corridor or plenum (4) and an air outlet opening or slot (5) inside the room to be treated. A ventilation and/or air conditioning system (10), connected to the air inlet duct (9), makes it possible to ensure throughout the volume of the air circulation corridor (4), a laminar air flow between the air inlet (9) and the air outlet slot (5). The air outlet opening (5) is arranged in the peripheral attachment profile (12), the latter forming a profiled element referred to as air supply. Application WO 2018/037184 describes an example of such a profile that can be implemented with the false ceiling structure (1) according to the object of the present disclosure.

According to the present disclosure, the false ceiling (1) comprises a plurality of light means (6) that are, in the example shown, fixed to the ceiling slab or wall (3) or suspended in the plenum (4).

In the embodiment shown, the plurality of light means (6) comprise UVC light sources (6) distributed on the ceiling wall (3) or suspended from the ceiling wall (3), and able to emit ultraviolet light. Preferentially, these UVC light sources (6) are lamps, for example, light-emitting diodes, of UVC type. These sources, having a germicidal function, thus allow the air to be sterilized when it passes through the air circulation corridor (4).

The first protective fabric (8) is treated with, for example, a composition making it possible to prevent the passage of UVCs. It thus protects the persons present in the premises by blocking UVCs.

Advantageously, a deflector (16) is positioned in line with or at the air outlet slot (5) so as to prevent any passage of the UVCs by the latter and thus ensure perfect protection of the occupants of the premises.

Advantageously, the false ceiling (1) comprises means blocking ultraviolet light emitted by the UVC light sources (6), in order to form a protective screen of the tensioned protective fabric (8). This protective screen is made in the example shown by an additional tensioned finishing fabric (2) (designated as second tensioned fabric 2) visible from the room and arranged below the first tensioned fabric (8). As shown, the second tensioned fabric (2) is arranged at a distance from the first tensioned fabric (8) and is visible from the premises. In the case where the false ceiling (1) comprises such a finishing fabric (2), the air inlet opens into the air circulation corridor (4), between the first tensioned fabric (8) and the ceiling wall (3). Of course, an air inlet is provided in the space delimited by the second tensioned fabric (2) and the first tensioned fabric (8). In this case, light sources may be provided on the wall in which the air inlet is arranged and on the wall near the air outlet slot (5) inside the room. As before, the air outlet openings (5) are provided both for the first tensioned fabric (8) and for the second tensioned fabric (2), at the smooth profiles (12) on which the two tensioned fabrics are fixed. The presence of the second tensioned fabric (2) is, of course, optional, and a false ceiling (1) without an additional fabric (2) can be provided. Likewise, a false ceiling (1) can be provided, comprising several additional protective-fabrics, advantageously arranged parallel to each other and at a distance from one another so as to define a plurality of air circulation passages. In this case, each profile or peripheral attachment (12) comprising an air outlet opening (5) in the adjoining underlying corridor. The smooth profiles (12) thus have air outlet openings (5) arranged relative to one another to define a downward circulation substantially vertical along the wall, until the air outlet opening (5) passes through the room. Furthermore, in the shown embodiment, the two fabrics are attached via common rail profiles (12). A separate profile of stringers may be provided, of course, for each of the fabrics without departing from the scope of the present disclosure.

As shown, air from the air suction openings (14) is blown by the ventilation or air conditioning system (10) into the circulation corridor or plenum (4) via the air inlet duct (9). As soon as it enters the air circulation corridor (4), the air flow rate is subjected to a flow section that is much greater than that of the air supply duct (9); the air speed then greatly decreases generating a laminar air flow entrained along a path substantially parallel to the ceiling wall (3), under the UVC light sources (6). The air is thus subjected to radiation from the UVC light sources (6) over the entire length of the circulation corridor and for a long period of time. By way of example, for a part of 20 m² (5 m×4 m) powered by 800 m³/h of treated air and for an air circulation corridor with a height of 30 cm, the air will pass from a conventional speed of approximately 2.5 m/s in the pipe (9) to 0.18 m/s as soon as it enters the air circulation corridor (4), or a reduction by a factor of 14. Thus, in this configuration, the minimum exposure time of an air molecule will be 28 seconds. By way of comparison, it would be 0.4 seconds for a sterilization device inserted over 1 m from the air supply duct (9). This arrangement has the advantage of guaranteeing the treatment of air, regardless of its flow rate. In fact, depending on the value of the air flow rate and the geometry of the part to be treated, it is possible to determine the height of the air circulation or plenum passage (4) so as to obtain the desired exposure time to UVC. The air then passes through the outlet opening of the first fabric (8) and then the outlet opening of the second fabric (2) so as to be diffused toward the room to be treated.

In the embodiment shown, the ceiling or false ceiling structure (1) comprises a tensioned ceiling. The present disclosure is not limited to such a ceiling structure, with the structures comprising suspended ceiling tiles, metal ceilings or plaster, suspended islets or any other type of ceiling falling within the scope of the present disclosure.

Aside from the sterilization of the air passing through the air circulation corridor (4), the present disclosure has the advantage of ensuring sterilization of all the solid bodies opening on the circulation corridor (4) such as the lower face of the ceiling (3), the vertical drops between the ceiling (3) and the first fabric (8) or any element that would be installed within the circulation corridor (4).

Advantageously, the surfaces of the plenum formed by the air circulation corridor (4) such as the underside of the ceiling (3) or even the vertical drops between the ceiling (3) and the first fabric (8) can be covered with a hygienic material such as a stainless steel sheet or a polymer coating (such as PVC, for example), in particular, for the uses in premises that are constrained to strong hygienic constraints, such as hospitals.

Advantageously, the hygienic material used to trim the plenum surfaces (4) is reflective thus avoiding any loss of UVC radiation emitted within the plenum by absorption of the hygienic material.

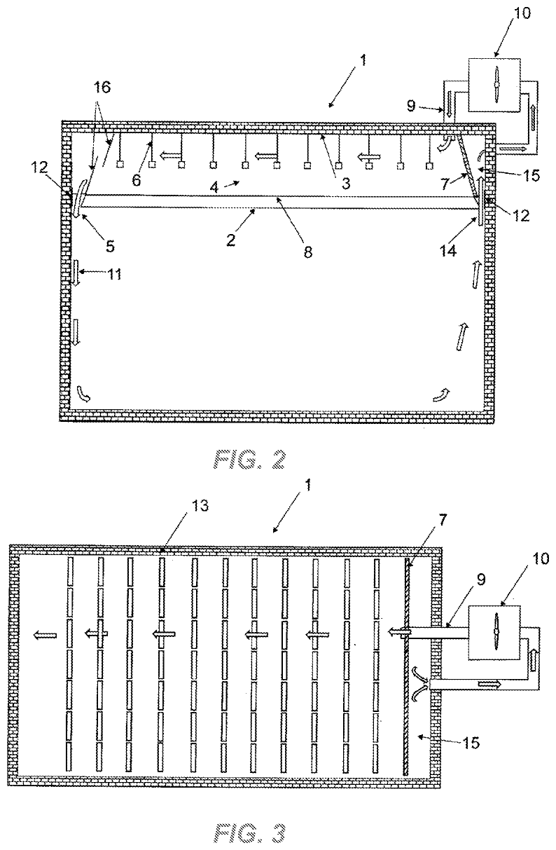

In a particular embodiment, as shown in FIGS. 1-4, the ceiling structure comprises both the blowing of air treated and sterilized to the room or room to be treated and the intake of indoor air to the air treatment unit. In this case, the air intake is carried out along a wall opposite the air blowing wall via an air suction opening (14) equipped with a casing of an air return (15).

The present disclosure is described in the foregoing by way of example. It is understood that a person skilled in the art is able to produce different variant embodiments of the present disclosure without departing from the scope of the present disclosure.

### EXAMPLES

### Example 1

Disinfecting efficacy calculations were conducted via numerical simulations (CFD) on the basis of a defined part of 4 m wide by 5 m in length with 2.5 m height under ceiling, that is under the tensioned finishing fabric (2).

The height of the plenum (4) is 20 cm between the natural ceiling (3) of the part and the tensioned protective fabric (8).

Other simulations have been carried out by varying the height of the plenum (4) to a total height of 1 m, so as to evaluate the impact of this parameter on the disinfection quality.

The air conditioning/ventilation unit (10) provides an air flow rate of 343 m³/h, corresponding to a thermal power of 70 W/m² on the ground, power commonly used for the purposes of cooling of buildings in France.

The air intake is carried out along a wall of 4 m via a 24 mm suction opening (14), a rectangular suction duct is connected to a suction box (15) composed of a separation partition (7) supported by the peripheral attachment profile (12).

The blowing of air treated by the air conditioning unit (10) into the plenum (4) by the rectangular network also called air ventilation (9) via 6 rectangular orifices of 300 mm×100 mm distributed over the width of the part.

The air outlet slot (5) to the part, with a width of 24 mm, is located on the small length of 4 m opposite the air suction opening (14).

UVC lamps (6) of OSRAM HNS 8 W G5 type of cylindrical tube type (diameter 16 mm and length 288 mm) are arranged in staggered rows along lines (13) at mid-height of the plenum (4), spacing of 50 cm between each line of lamps (13) or 9 lines of UVC lamps distributed over the length of 5 m, 7 UVC lamps (6) per line (13).

Another configuration was tested comprising 3 lines (13) of UVC lamps (6) or one line every 1.25 m.

Each UVC lamp (6) has a 2 W UVC transmission unit power according to the manufacturer's data.

Air disinfection thresholds used, also called dosages, for the simulations, that is the energy that an air molecule must receive in order to be freed of x % of the viruses and bacteria in the air were transmitted by the manufacturer of UVC Osram lamps. These thresholds are expressed in surface energy or the power received by an air molecule multiplied by the exposure time:
Assay of 2.6 mJ/cm 2 for 90% disinfection,
Assay of 5.0 mJ/cm 2 for 99% disinfection,
Assay of 8.8 mJ/cm 2 for 99.9% disinfection.

FIG. 5 presents a horizontal section resulting from the result of the numerical simulations. This section is located at the level of the light sources (6) installed in the plenum (4).

The modeling has, in particular, the behavior of the flow of air blown into the plenum by viewing the air speeds.

There is, in particular, a distinction between the lines (13) of UVC light sources (6), the UVC light sources (6) arranged in anyone, the air suction box (15), the air suction opening (14), and the air outlet slot (5).

The air inlet air venting duct (9) ejects the blown air into the plenum (4) via 6 ports directed toward the right side of the plenum.

This model that made it possible to determine the efficiency results described below makes it possible, in particular, to observe that the air flow is transformed into laminar flow during its journey into the plenum, changing at very slow speeds and therefore subjected to a significant exposure time to UVC radiation emitted by the light sources. Thus, the geometry of the false ceiling structure (1) according to the present disclosure imposes a much longer exposure time and therefore improved sterilization efficiency.

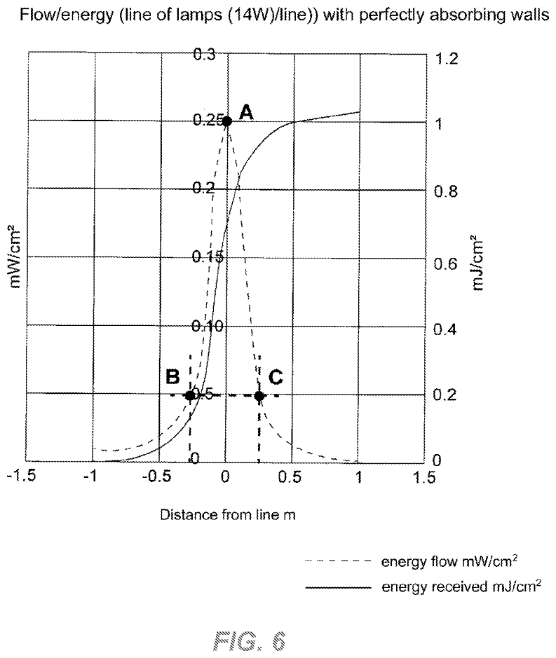

FIG. 6 presents the impact of the height of the plenum (4) on the disinfection quality.

Indeed, the disinfection quality is directly related to the quantity of energy in mJ/m2 received by the air flow in the presence in the plenum.

This curve gives the x-axis the distance between an air molecule and a line (13) of 7 UVC light sources (6). On the y-axis, the amount of energy received by an air molecule depending on the distance between this molecule and the line of light sources is obtained.

The curve adopts the shape of a Gauss curve. Point A represents an air molecule located against the line (13) of UVC light sources.

Point B represents an air molecule located 25 cm under the line (13) of UVC light sources.

Point C represents an air molecule located 25 cm above the line (13) of UVC light sources (6).

The result of the simulations gives an energy quantity of precisely 1.02 mJ/m² received by point A.

Thus, the farther the light sources (6) are, the higher the amount of energy received by the air and therefore the disinfection quality decreases.

It can be seen, in particular, that if it is desired to keep a reasonable part (20%) of the energy emitted by the line (13) of light sources, air must not be able to pass by more than 25 cm below or above the line (13) of the UVC light sources (points B and C), which corresponds, with the line of UV light sources ideally placed at mid-height of the plenum (4), to a maximum plenum height of 50 cm.

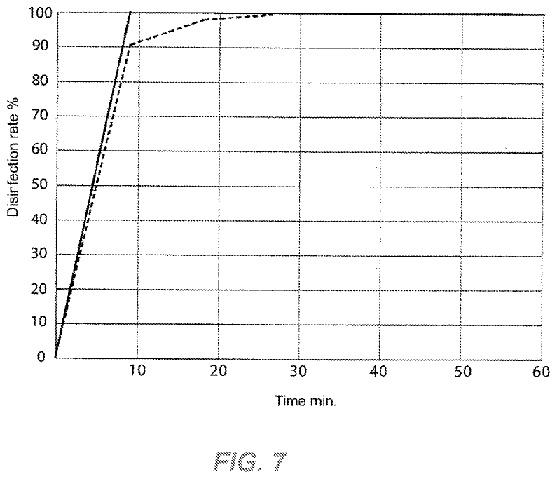

FIG. 7 represents the degree of disinfection in % of the interior air of the room or local to be treated as a function of the operating time of the light sources (6) and therefore of the system.

Two configurations were calculated:
9 lines (13) of light sources spaced apart by 50 cm for the continuous curve,
3 lines (13) of light sources spaced from 1.25 m for the dotted curve.

The calculations were carried out for a plenum (4) of 20 cm.

It can be seen that for 9 lines of light sources (6), less than 8 to 9 minutes of operation of the ventilation system (10) are necessary to achieve a 99.9% disinfection rate of the interior air of the component and that for 3 lines of light sources (6), less than 27 minutes of operation of the ventilation system (10) are necessary to achieve a 99.9% disinfection rate of the interior air of the part, independently of the number of occupants of the component.

In conclusion, the system thus tested has by the geometry of the plenum (4) a laminar flow of the air flow within the latter having the advantage of a long exposure time to the UVC flow emitted by the plurality of light sources (6).

The height of the plenum limited to 50 cm makes it possible to benefit from at least 20% of the UVC radiation emitted by the light sources (6) making it possible to limit the number and power of the latter.

With a spacing between the lines (13) of UVC light sources (6) of between 50 cm and 1.25 m, a reduced operating time of the system (between 8-9 and 27 minutes) makes it possible to achieve a degree of disinfection of the inside air of the part by 99.9%.

### REFERENCE NUMBERS EMPLOYED IN THE FIGURES

1: False ceiling
2: tensioned finishing fabric
3: ceiling of the room to be sterilized
4: so-called plenum space
5: air outlet slot
6: plurality of UVC light sources
7: separation partition
8: protective tensioned fabric
9: air inlet aeraulic ventilation
10: ventilation and/or air conditioning means
11: supply of treated air
12: peripheral hook profile
13: dials of light lamps
14: air intake openings
15: air suction channel also called air return box
16: deflector.

1. A sterilization system for sterilizing a room, comprising:
a false ceiling for supplying treated or not treated air in the room to be sterilized;
at least one protective tensioned fabric, blocking UVC, the at least one protective tensioned fabric positioned on the underside and at a distance from the ceiling of the room to be sterilized, being fixed to the side walls of the room and arranged with an upper slab of the ceiling in order to delimit a so-called plenum space;
at least one means for attaching the at least one protective tensioned fabric to the side walls of the room by means of at least one peripheral attachment profile comprising an air outlet slot opening onto at least one side wall of the room and allowing the passage of the air circulating between the plenum and the room to be sterilized;
—at least one ventilation and/or air-conditioning means connected to the plenum via at least one air inlet air venting duct in the plenum, the blown air forming a horizontal unidirectional air circulation corridor over the entire length of the plenum;
—an air return box able to be connected to ventilation and/or air conditioning means, as well as air suction openings located opposite the air outlet slot and allowing air to pass from inside the room to the air return box; and
several UVC light sources arranged in the plenum over the entire length of the horizontal unidirectional air circulation corridor so as to sterilize the air flowing in the air circulation corridor before it passes through the air outlet slot, wherein a distance between the plurality of UVC light sources and the ceiling on the one hand and a distance between the plurality of UVC light sources and the at least one tensioned protective fabric on the other hand is at most 25 cm, and wherein the walls of the plenum have means of reflection of ultraviolet light emitted by the UVC light sources.

2. The system of claim 1, wherein the false ceiling further comprises at least one tensioned finishing fabric, the tensioned finishing fabric, visible from the room, being fixed to the side walls of the room by means of the at least one peripheral attachment profile comprising an air outlet slot opening onto at least one side wall of the room and allowing the passage of the air circulating between the plenum and the room to be sterilized.

3. The system of claim 1, wherein a height of the plenum is at most 50 cm.

4. The system of claim 3, wherein the height of the plenum is at least 10 cm.

5. The system of claim 1, wherein the plurality of UVC light sources are arranged at mid-height of the plenum.

6. The system of claim 1, wherein the UVC light sources are arranged in the form of lines of light lamps that are spaced apart over an entire length of the plenum.

7. The system of claim 6, wherein the lines of light lamps are continuous or discontinuous lines.

8. The system of claim 7, wherein, wherein the lines of light lamps comprise staggered rows of discontinuous lines.

9. The system of claim 6, wherein a space between two adjacent lines of light lamps is between 25 cm and 200 cm.

10. The system of claim 9, wherein the space between the two adjacent lines of light lamps is between 50 cm and 100 cm.

11. wherein the air of the room to be sterilized has a disinfection coefficient of 99.9% of the viruses and bacteria when the ventilation and/or air conditioning means is activated for at most 30 minutes and this independently of the number of occupants in the room to be sterilized.

12. The system of claim 1, wherein the air of the room to be sterilized has a disinfection coefficient of 99.9% of the viruses and bacteria when the ventilation and/or air conditioning means is activated for at least 8 minutes and this independently of the number of occupants in the room to be sterilized.

13. (canceled)

14. The system of claim 1, wherein a face of the protective tensioned fabric facing onto the plenum has a UVC reflection coefficient of greater than or equal to 80%.

15. The system of claim 2, wherein the protective tensioned fabric and the finishing fabric each have a thickness of less than 5 mm.

16. The system of claim 1, wherein a suction channel is defined by a separation partition integral with the peripheral attachment profile.

17. An installation for supplying treated air to a room to be sterilized, comprising:
the system according to claim 1;
an air inlet in the plenum, an air outlet slot of the false wall being arranged at the opposite end of the air inlet, and
ventilation and/or air conditioning means connected to the air inlet in order to form a horizontal unidirectional air circulation corridor of the plenum;
wherein the walls of the plenum include means for reflecting ultraviolet light emitted by a plurality of UVC light sources.

18. The installation according to claim 17, wherein the ventilation and/or air conditioning means are connected to the air return box.
